(19) United States
(12) Patent Application Publication
(10) Pub. No.: US 2005/0265937 A1
(43) Pub. Date: Dec. 1, 2005

(54) Systems and methods for changing eye color appearance

(76) Inventor: Alan Glazier, Rockville, MD (US)

Correspondence Address: BERENATO, WHITE & STAVISH, LLC, 6550 ROCK SPRING DRIVE, SUITE 240, BETHESDA, MD, 20817, US

(21) Appl. No.: 10/926,964

(22) Filed: Aug. 27, 2004

Related U.S. Application Data
(60) Provisional application No. 60/575,746, filed on Jun. 1, 2004.

(51) Int. Cl. A61K 7/021; A61K 39/395

(52) U.S. Cl. 424/63; 424/178.1

(57) ABSTRACT

Methods and systems are provided for changing the appearance of eye color. The methods and systems generally comprise administering a biologically compatible molecule (preferably a coloring agent) coupled to an antibody to an eye of a subject, and binding the antibody to a structure, preferably the cornea of the eye.

### RELATED APPLICATIONS

The application claims the benefit of priority of provisional application 60/575,746 filed in the U.S. Patent & Trademark Office on Jun. 1, 2004, the disclosure of which is incorporated herein by reference.

### BACKGROUND OF THE INVENTION

1. Field of the Invention

The present invention generally relates to a method for changing the appearance of eye color, preferably for a human eye.

2. Description of the Related Art

It is desirous among certain people to change the color of their eye, in particular the appearance of the iris region of their eye. This change is currently achieved through use of cosmetic contact lenses. For instance, people with brown eyes can “cover” their natural iris with a tinted contact lens. The cosmetic tint manufactured within the tinted contact lens is placed directly on the cornea of the user. The contact lens either additively changes color (as in “enhancement”), or, if the intention is to change a darker eye color, alters eye color by adding a lens that is opaque and blocks out the underlying iris color, replacing it with the color of the contact lens. Opaque lenses have a hole in the center so light intensity is not diminished as the light passes through the lens into the user's eye via the pupil.

Such contact lenses present various potential drawbacks, including the possibility of infection, dryness and irritation, inconvenience of periodic lens removal and cleaning, and lens fragility.

### SUMMARY OF THE INVENTION

It is an object of the invention to provide a method and system for effectively changing the appearance of color of the eye (or “eye color”).

It is another object of the invention to provide a method and system for changing the appearance of eye color in a simple and easy to administer manner.

It is still another object of the invention to provide a method and system for changing the appearance of eye color while overcoming the drawbacks of conventional contact lenses.

In accordance with the purposes of the invention as embodied and broadly described in this document, and to attain one or more of the above-discussed desirable objects, methods and systems are provided for changing the appearance of eye color. The methods and systems generally comprise steps and means for administering a biologically compatible molecule (or coloring agent) coupled to an antibody to an eye of a subject, and binding the antibody to a structure, preferably the cornea of the eye.

An aspect of the invention provides methods and systems for changing the appearance of eye color by administering biocompatible molecules (or coloring agent) to the cornea of the eye. The biocompatible molecules are conjugated to antibodies that are delivered and bind to the cornea, preferably a constituent (or component) of the cornea selected from ocular collagens, proteins and/or other molecules (e.g., glycos amino glycans (GAGs), proteoglycans) found on or within layers of the cornea.

The methods and systems of certain aspects and embodiments of the invention provided herein preferably deliver biocompatible molecules to the eye via antibodies designed to attach to unique ocular species within the eye. By conjugating molecules to the antibodies, the antibodies act as a delivery system engineered to bind to specific molecules located in a selected one or plurality of ocular structures. The eye has several proteins, collagens, and/or other molecules, tissues, and membranes that are unique to the eye and, in some cases, unique in their location in the eye in respect to other proteins, specifically collagens in the eye and in the subject's body. The specificity of the antigen-antibody reaction, coupled with inherent biological variation, makes an antibody a highly specific reagent for identifying individual molecules in complex mixtures, and distinguishing related molecules.

Specifying the antibodies to bind to unique ocular proteins, collagens, and/or other molecules of various structures, membranes, and tissues may result in one or more of the following advantages. First, this technique offers a direct avenue for drug delivery. Second, as the antibodies are customized to bind to molecules found specifically in the eye, the antibodies should not attach to non-intended tissues and cause undesirable changes or side effects for the user. Third, therapy may be applied easily with a non-intrusive technique, such as an eye drop or intra-ocular injection.

### DETAILED DESCRIPTION OF PREFERRED EMBODIMENTS AND PREFERRED METHODS OF THE INVENTION

Reference will now be made in detail to the presently preferred embodiments and methods of the invention. It should be noted, however, that the invention in its broader aspects is not limited to the specific details, representative systems and methods, and examples shown and described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

The term “conjugated” and variations thereof are to be interpreted in the broadest sense, unless the context clearly dictates otherwise, to include binding, attaching, joining, or coupling, etc.

The term “biocompatible” comprises a material or materials generally understood as biologically compatible with the subject's eye. In particular, the materials are preferably substantially non-toxic, substantially non-hemolytic, and substantially non-irritant.

The terms “subject” and “patient” are used generally interchangeably herein, and may refer to a human or animal unless the context clearly dictates otherwise, e.g., a “human subject”.

The phrases “appearance of color of the eye” and “appearance of eye color” are meant with reference to the view of a person of ordinary eyesight viewing the treated eye bathed in light from the visible light spectrum, e.g., approximately 700 nm to approximately 400 nm wavelength.

In the below described embodiments, the biocompatible molecules may be administered to the eye using any suitable method, unless clearly stated otherwise. Preferably, the administration method is topical, e.g., with an eye drop. Injection is also possible in various embodiments. Although not necessarily specified below in the following embodiments, it is possible to use other administering methods in several of the below-described embodiments, such as adenoviral vector, lipid vector such as lipid microspheres, electro-ionic transport, contact lens as therapeutic delivery system, slow drug release technology, and other techniques. The techniques are preferably performed in vivo, although in vitro procedures are also contemplated.

The human cornea comprises several layers. Each layer is composed of a different structure comprised of collagen, with the collagen types varying from layer to layer. The corneal layers comprise and are arranged, from anterior to posterior in direction, in the following order: epithelium; epithelium basement membrane; Bowman's membrane; stroma; Descemet's membrane; and endothelium.

The epithelial basement membrane (EPBM), also known as the basal lamina, is composed of two layers, the lamina lucida and the lamina densa. The EPBM is composed of primarily Type 7 collagen but also has some Type 6. Other components include laminin, heparan sulfate proteoglycan, fibronectin and fibrin.

Bowmans layer, located immediately behind the EPBM, is approximately 8-12 micrometers thick and made of randomly arranged collagen fibrils. The Bowmans layer is made of Types 1, 3, 5, and 6 collagen.

The corneal stroma is a dense connective tissue of remarkable regularity. The corneal stroma makes up the vast majority of the cornea and predominantly comprises a 2 μm thick flattened collagenous lamellae (200-250 layers) orientated parallel to the corneal surface and continuous with the sclera at the limbus. Between the lamellae lie extremely flattened, modified fibroblasts known as keratocytes. The collagen fibers are predominantly of Type 1 (30 nm diameter, 64-70 nm banding), with some Type 3, 5 and 6 and 12. The transparency of the cornea is highly dependent upon the regular spacing of the collagen fibers (interfibrillary distance), which in turn is regulated by glycosaminoglycans (GAG) and proteoglycans forming bridges between the collagen fibrils. The GAGs in the human cornea are predominantly keratan sulphate and chondroitin (dermatan) sulphates.

Descemet's membrane is composed of Type 4, 5, 6, 8, but primarily Type 4 collagen, laminin and fibronectin. This is a thin, homogenous, discrete, PAS-positive layer between the posterior stroma and the endothelium, from which it can become detached. Descemet's membrane is 8-12 μm (microns) in thickness and represents the modified basement membrane of the corneal endothelium. It possesses two parts: an anterior third which is banded and a homogeneous or non-banded posterior two-thirds. The Descemet's membrane is rich in basement membrane glycoproteins as well. The anterior banded region is reported to contain Type 8 collagen. Types 5 and 6 collagen may be involved in maintaining adherence at the interface of Descemet's membrane with the most posterior lamellae of the stroma. It may be less desirable in certain instances to tint Type 1 collagen, because the sclera is primarily Type 1 collagen and tinting may leach onto the white part of the eye and not be retained solely by the cornea, causing a poor appearance.

According to an embodiment of the invention, a method and system are provided for delivering biocompatible molecules targeted to the cornea of the eye. The biocompatible molecules are conjugated to antibodies selected to bind to specific constituents of the cornea, preferably specific ocular collagens, proteins and/or other molecules found within layers of the cornea. The biocompatible molecules of the first embodiment preferably comprise a coloring agent selected from dyes, stains, pigments, and other molecules capable of absorbing and/or reflecting light within layers of the cornea to change the outward appearance, especially the color, of the eye.

The corneal epithelial basement membrane, Bowman's membrane, the corneal stroma, descemet's membrane, and epithelium, either alone or in any combination, are preferred for targeting. The particular membrane (or structure) of the cornea subject to targeting may be predetermined by basing the selection of the antibody on its ability to bind to different membranes, i.e., to bind to collagen-type found in a certain membrane but not others.

Generally, binding to Type 7 collagen is highly preferred. Type 7 collagen is found only in the epithelial basement membrane, but is not found in any significant amounts in the other membranes of the cornea, the sclera, or conjuctiva. Other preferred binding sites include collagen type 6 and type 12. The antibody may also be specific, for example, to 64 kilo Dalton keratin of the cornea epithelium.

Corneal structures of superficial layer(s) of the cornea that antibodies (with conjugated molecules) may bind to for changing the appearance of color of the superficial layers of the cornea are preferably anterior to the stroma. Binding sites include, for example and not necessarily by limitation, the following: Type 7 collagen, fibrin, fibronectin, heparin sulfate and/or laminin. Corneal structures of stroma(s) of the cornea that antibodies (with conjugated molecules) may bind to for changing the appearance of color of the superficial layers of the cornea according to this first embodiment include, for example and not necessarily by limitation, the following: chondroitin, chondroitin sulfate A, keratan sulfate, Type 3 collagen, Type 1 collagen, Laminin, Type 6 collagen, Type 5 collagen, Type 12 collagen, Laminin-1, and/or Laminin-5.

The biologically compatible molecule of the first embodiment is preferably a vegetable dye, vital dye, pigment, or the like, although other biologically compatible molecules may be used in connection with this embodiment. Specific examples of such molecules include, for example and not necessarily limitation, alumina (dried aluminum hydroxide); annatto extract; calcium carbonate; canthaxanthin; caramel; β-carotene; cochineal extract; carmine; potassium sodium copper chlorophyllin (chlorophyllin-copper complex); dihydroxyacetone; bismuth oxychloride; synthetic iron oxide; ferric ammonium ferrocyanide; ferric ferrocyanide; chromium hydroxide green; chromium oxide greens; guanine; pyrophillite; mica; talc; titanium dioxide; aluminum powder; bronze powder; copper powder; zinc oxide; FD&C blue No. 1; FD&C blue No. 2; D&C blue No. 4; FD&C green No. 3; D&C green No. 5; D&C green No. 6; D&C green No. 8; D&C orange No. 4; D&C orange No. 5; D&C orange No. 10; D&C orange No. 11; FD&C red No. 3; FD&C red No. 4; D&C red No. 6; D&C red No. 7; D&C red No. 17; D&C red No. 21; D&C red No. 22; D&C red No. 27; D&C red No. 28; D&C red No. 30; D&C red No. 31; D&C red No. 33; D&C red No. 34; D&C red No. 36; D&C red No. 39; FD&C red No. 40; D&C violet No. 2; FD&C yellow No. 5; FD&C yellow No. 6; D&C yellow No. 7; ext. D&C yellow No. 8; D&C yellow No. 10; D&C yellow No. 11; bismuth citrate; disodium EDTA-copper guaiazulene; henna; lead acetate; silver; ultramarines; manganese violet; luminescent zinc sulfide; D&C brown No. 1; chromium-cobalt-aluminum oxide; ferric ammonium citrate; pyrogallol: C.I. oxidation base 32; logwood extract; C.I. natural black 1; 1,4-bis[(2-hydroxy-ethyl)amino]-9,10-anthracenedione bis(2-propenoic)ester copolymers; 1,4-bis [(2-methylphenyl)amino]-9,10-anthracenedione; 1,4-bis[4-(2-methacryloxyethyl) phenylamino] anthraquinone copolymers; carbazole violet; C.I. vat orange 1, 2-[(2,5-diethoxy-4-[(4-methylphenyl)thiol] phenyl)azo]-1,3,5-benzenetriol; C.I. vat brown 1: 16,23-dihydrodinaphtho [2,3-a:2′,3′-i] naphth [2′,3′:6,7] indolo [2,3-c]carbazole-5,10,15,17,22,24-hexone; C.I. vat yellow 3: N,N′-(9,10-dihydro-9,10-dioxo-1,5-anthracenediyl) bisbenzamide; C.I. vat blue 6: 7,16-dichloro-6,15-dihydro-5,9,14,18-anthrazinetetrone; C.I. vat green 1: 16,17-dimethoxydinaphtho (1,2,3-cd:3′,2′,1′-lm) perylene-5,10-dione; poly(hydroxyethyl methacrylate)-dye copolymers (e.g., C.I. reactive black 5, C.I. reactive blue 21, C.I. reactive orange 78, C.I. reactive yellow 15, C.I. reactive blue 19 C.I. reactive blue 4, C.I. reactive red 11, C.I. reactive yellow 86, C.I. reactive blue 163, and/or C.I. reactive red 180), C.I. solvent yellow 18: 4-[(2,4-dimethylphenyl)azo]-2,4-dihydro-5-methyl-2-phenyl-3H-pyrazol-3-one; C.I. vat orange 5: 6-ethoxy-2-(6-ethoxy-3-oxobenzo(b) thien-2 (3H)-ylidene) benzo(b)thiophen-3 (2H)-one; phthalocyanine green; vinyl alcohol/methyl methacrylate-dye reaction products (e.g., C.I. reactive red 180, C.I. reactive black 5, C.I. reactive orange 78, C.I. reactive yellow 15, C.I. reactive blue 19, and/or C.I. reactive blue 21); D&C blue No. 9, (phthalocyaninato(2-)) copper; D&C blue No. 6; C.I. vat orange 1; and/or phenyl]azo]-1,3,5-benzenetriol. These molecules may be used alone or in combination with one another or other molecules.

The concentration of coloring agent-antibody conjugate should be sufficient to effect the desirable degree of tint to the cornea. The selected concentration will depend upon various factors, including the selected coloring agent, the natural color of the subject's eye, and the desired tint. For example, in the case of a subject having a very light eye color and seeking a slight enhancement to their natural eye color of about 20%, the concentration of antibody to antigen (in the human cornea) might be approximately 1:5. For attaining a more opaque tint, the concentration of antibody to antigen might approach 1:1.

If the cornea is tinted at the epithelial basement membrane or the Bowman's layer or upper ½ of stroma, it may be desirable (but optional) to use a laser, such as an excimer laser, to destroy the membrane that exists within the pupillary line of sight. The recipient (or subject) is thereby provided with a “hole” centrally in the tinted cornea that the recipient can look through without color tainting his/her line of sight.

The biocompatible molecules may be administered to the cornea using any suitable method. Preferably, the administration method is topical. It is possible to use other administering methods, such as injection, adenoviral vector, and other techniques, some of which are mentioned above. A flap of cornea may be lifted and tint applied underneath as well.

The antibodies of the various embodiments may be polyclonal or monoclonal antibodies, and may also comprise molecules that are fragments and derivatives of such antibodies, including, for example, F(ab′)₂, Fab′ and Fab fragments. Such antibodies may be monospecific, or may comprise bispecific antibodies, such as chimeric antibodies, hybrid antibodies, etc. having at least two antigen or epitope binding sites. Monoclonal antibodies are preferred.

Methods for isolating or obtaining such immunoglobulins are well-known in the art (Kohler, G. & Milstein, C., Nature 256:495-497 (1975); Taggart & Samloff, Science 219:1228-1230 (1983); Kozbor et al., Immunology Today 4:72-79 (1983); Morrison et al., Proc. Natl. Acad. Sci. USA 81:6851-6855 (1984); Takeda et al., Nature 314:452-454 (1985)); Biocca, S. et al., EMBO J. 9:101-108 (1990); Bird, R. E. et al., Science 242:423-426 (1988); Boss, M. A. et al., Nucl. Acids Res. 12:3791-3806 (1984); Boulianne, G. L. et al., Nature 312:643-446 (1984); Bukovsky, J. & Kennett, R. H., Hybridoma 6:219-228 (1987); Diano, M. et al., Anal. Biochem. 166:223-229 (1987); Huston J. S. et al., Proc. Natl. Acad. Sci. USA 85:5879-5883 (1988); Jones, P. T. et al., Nature 321:522-525 (1986); Langone, J. J. & Vunakis, H. V. (Editor), Methods Enzymol. 121, Academic Press, London (1987); Morrison, S. et al., Proc. Natl. Acad. Sci. USA 81:6851-6855 (1984); Oi, V. T. & Morrison, S. L., BioTechniques 4:214-221 (1986); Riechmann, L. et al., Nature 332:323-327 (1988); Tramontano, A. et al., Proc. Natl. Acad. Sci. USA 83:6736-6740 (1986); Wood, C. R. et al., Nature 314:446-449 (1985); and Ladner, U.S. Pat. No. 4,946,778, issued Aug. 7, 1990.

Polyclonal antibodies may be produced through any of a variety of well known methods. For example, various animals may be immunized for this purpose in known manner by injecting them with an antigen (for example a lens protein, collagen, or crystallin, or another molecule sharing an epitope of such molecules). Such antigen molecules may be of natural origin or obtained by DNA recombination or synthetic methods, or may comprise fragments thereof. Desired polyclonal antibodies can be recovered from the resulting sera and purified by known methods. Alternatively, intact cells that array the antigen molecule may be used. Various adjuvants may also be used for increasing the immune response to the administration of antigen, depending on the animal selected for immunization. Examples of these adjuvants include Freund's adjuvant, mineral gels such as aluminum hydroxide, surfactant substances such as polyanions, peptides, oil emulsions, haemocyanins, dinitrophenol or lysolecithin.

There are several accepted methods of monoclonal antibody production, which typically include the step of isolating the antigenic target of the antibody. Methods of isolating antigen are standard practice and well understood in the art. The following materials and methods are presented by way of example and illustration, and are not necessarily limiting upon the scope of the invention and its various embodiments.

Cell Culture Medium

The two culture media used in most laboratories are Dulbecco's minimum essential medium (DMEM) and RPMI 1640. The powder form is sterilized once dissolved. The quality of water is important. Water purification units for different purposes produce different quality water; the unit used should be intended for tissue culture. Medium is conveniently used in 500 ml aliquots and stored at 4° C. before use. Medium should be warmed to 37 degrees C. prior to use. To the medium add 2 mM glutamine, 100 IU ml⁻¹ penicillin, 100 mg ml⁻¹ streptomycin and fetal bovine serum (FBS) to 10%. If the medium is stored for more than 2 weeks at 4° C., the levels of the essential amino acid glutamine, and the antibiotics penicillin and streptomycin should be replenished.

Fetal Bovine Serum (FBS)

Serum is used to provide additional nutrients to the medium to support cell growth. FBS is still the most commonly used serum additive for tissue culture media. Different batches of FBS support cell growth to different degrees. Batches of FBS can be screened for ability to support hybridoma growth, although prescreened batches are available commercially. Mixtures of sera have been used for hybridoma culture, and the addition of mouse serum has been reported to increase hybridoma yields. A-gamma (Ig depleted) calf serum has been shown to produce twice as much immunoglobulin as standard FBS, in both human and mouse hybridoma lines (Torres et al., 1992), and purification of monoclonal antibody subsequently is easier if there is no bovine IgG present.

Serum-Free Media

The risk of introduction of pathogens such as bovine viruses or prions (which cause diseases such as bovine spongiform encephalopathy and Cfreutzfeld-jacob syndrome) and the presence of unwanted proteins in downstream processing has generated the desire to use completely defined serum-free medium.

Selection Medium

Medium containing hypoxanthine, aminopterin and thymidine (HAT) is used to selectively grow hybrids following fusion. Aminopterin blocks the main biosynthetic pathway for DNA synthesis, while thymidine and hypoxanthine feed the salvage pathways. For each fusion, a fresh bottle of HAT medium should be made by the addition of 100×stock HAT to the culture medium. A useful amount is about 100 ml of HAT medium/10⁸ lymphocytes fused. Medium containing hypoxanthine and thymidine (HT) is used to maintain hybridoma growth. Because hypoxanthine and thymidine are used up by cells in culture whilst aminopterin is not, cells will die unless HT medium is added, until the aminiopterin has been diluted out or removed. From 7 days following the fusion, the hybridomas are maintained in medium with HT. Some laboratories wean hybridomas off HT medium once the aminopterin is depleted, but the effort involved outweighs the savings in HT.

Lysis Medium

If spleen cells are used as the source of immune cells for fusion, the erythrocytes are commonly lysed prior to fusion, using isotonic ammonium chloride or Gey's hemolytic medium. However, this step is not essential and can compromise the quality of the lymphocytes.

Polyethylene Glycol (PEG)

PEG is the fusion-inducing agent. Batches of PEG vary in their toxicity and ability to induce fusions. PEG should be sorted in the dark, to avoid degradation by photooxidation. Some groups add dimethylsulfoxide (DMSO, 15% (v/v)) to the PEG for fusion, but the value of DMSO in the fusion process is questionable.

Screening Assay

One of the keys to successful development of a monoclonal antibody is the screening assay. The more specific and simple the screening test, the better the chance of obtaining a monoclonal antibody of interest. The nature of the antigen will often dictate the screening assay. For example, antibodies to surface antigens of cells in suspension can be examined quickly and easily by immunofluorescence, whereas immunoenzyme techniques are suitable for tissue sections and enzyme-linked immunosorbent assay (ELISA) or radioimmunoassay (RIA) for soluble antigens. Antibodies that react against fixed tissue will not necessarily react with fresh tissue. It is important to have purified antigen for the assay, since antibodies against impurities in the immunizing material will react if the test material contains the same impurities. The assay is preferably specific, sensitive and capable of screening large numbers of samples quickly. Appropriate positive and negative controls are in every assay.

Immunization

Selection of Antigen

As much as 1 mg of antigen may be required for the immunization and screening. The antigen should be as pure as possible because there will be an immune response against contaminants in the preparation. The purity of the antigen used in the detection assay is important; methods of screening for antibody against a component of a mixture, such as western immunoblotting or biological assays, are considerably more labor intensive than ELISA. The protein should be no smaller than 3 kDa, and should differ in amino acid sequence from the corresponding endogenous protein, in order to induce an immune response. Smaller or endogenous molecules can be made immunogenic by conjugation to a carrier protein, such as diphtheria toxoid.

Synthetic peptides that correspond to an amino acid sequence of the antigen can be prepared or purchased for use as antigen. The peptide should correspond to a sequence that is present on the exterior of the antigen molecule, and should be predicted to be antigenic, on the basis of the literature or antigenicity programs such as MacVector. If the synthetic peptide is small it should be conjugated to a carrier protein.

Standard Immunization Protocol

The response to soluble antigens is greatly potentiated by using an adjuvant. Complete Freund's adjuvant (CFA) is efficient and still popular. Other adjuvants include GMDP (GERBU Adjuvant 01, GERBU Biotechnik, Gailberg, Germany).

A booster immunization is commonly given about 2-4 weeks following the primary immunization; this should not be given in CFA because the risk of anaphylactic shock, but may be given in incomplete Freund's adjuvant (which is the same as CFA except that the mycobacterial component is missing). Preferably, the same aqueous adjuvant as for the primary immunization is used.

Antibody titers greater than 1 in 100 mouse serum is the minimum required to consider using the splenocytes in a fusion. If the mouse was immunized with an impure preparation of antigen and then screened with the same material it is necessary to confirm a specific immune response against the antigen, for example, by western blotting to identify the antigen by molecular weight, or by inhibition of the antigen's biological activity.

Sample Procedure

6 week old female mice>immunize each mouse with 50 micrograms antigen>wait 2 weeks>immunize each mouse with 50 micrograms antigen>wait 2 weeks>collect serum from mice and determine polyclonal titer>is the serum titer sufficient for fusion?—yes, then you have fusion—no, go back to immunizing mice.

Alternative Immunization Protocols

Several techniques have been used to overcome the problems of very low levels of antigen, including in vitro immunization, intrasplenic immunization and lymph node deposition. Small amounts of antigen purified on nitrocellulose membranes have been used to immunize animals either by an intrasplenic route or in vitrol. These methods generally result in monoclonal antibodies of the IgM isotype and often of low affinity.

Myeloma Cells

A Number of Myeloma Cell Ines are Available for Fusion.

| CELL LINE | REFERENCE |
|---|---|
| SP2/0Ag14 | Shulman et al., 1978 |
| P3-X63-Ag8.653 | Kearney et al., 1979 |
| FO | De St. Groth and Scheidegger, 1980 |
| P3-NS1/1-Ag4-1 (secretes Kappa | Kohler et al., 1976 |
| chain) | |

Rat, mouse, chicken, hamster and other mammals may be used as fusion partner.

The maintenance and health of the myeloma fusion partner is of importance in the eventual success of the fusion. Fresh myeloma cells are preferable to ones that have been growing a long time.

Fusion Protocol

Established fusion protocols use PEG to induce membrane fusion. Electroporation and electroacoustic techniques are alternative that are especially useful when low numbers of specific B cells are available for fusion. When combined with in vitro immunization methods, the purification of antigen-specific B cells followed by electroporation enables the production of monoclonal antibodies against low amounts of antigen.

There are many variations of the standard fusion protocol that may be used.

Post Fusion Care

Seven days after plating out the cells in HAT medium, replace half the medium with fresh medium containing HT, instead of HAT. At this time, small colonies of hybridoma cells may be visible. As the colonies grow, withdraw medium and test for antibody activity in the screening assay. Replace medium in the wells with fresh HT medium, as the medium becomes acidic (yellow).

### EXAMPLE PROCEDURE

1 week>inspect wells, identify hybridomas and mark these wells>replace medium if 2 weeks have elapsed since last replacement>is the medium sufficiently conditioned for screening>NO—go back to beginning. YES—screen the conditioned medium on 2 separate occasions>aspirate conditioned medium and replace with fresh medium>do any of the hybridomas test positive to antigen? NO>have all hybridomas been screened twice?>NO—go back to 1 week>YES—dispose of negative hybridomas>. If hybridomas test positive for antigen, clone and expand positive hybridomas.

Cloning

Once the screening assay indicates that a well contains an antibody of interest, the contents of the well should be cloned as soon as possible. It is important to clone positive wells so as to prevent them being overgrown by negative clones, and to avoid working with mixed clones. While there are several cloning methods, the most common is that of limiting dilution.

Cryopreservation

Preserving cells in liquid nitrogen is the only means of ensuring the long term availability of hybridomas. Cells should be frozen down as soon as possible and detailed record kept of what the cells are and when they were stored.

The remaining steps are well known in the literature and include
1. specificity and isotyping.
2. Mycoplasma contamination detection.
3. Large scale antibody production via Ascitic fluid, cell factory, perfusion cell culture.
4. Antibody purification including precipitation, chromatography and/or use of Protein G.
5. Storage and quality control.
   Conjugation of Antigen to a Carrier Protein
   Materials
Carrier protein (e.g. diphtheria toxoid). The mass ratio of carrier protein to antigen should be 4:1.
Glutaraldehyde solution (0.13 M).
Dialysis membrane with a molecular weight cut off that will allow unconjugated hapten to dialyse out but retain conjugate.
TBS buffer (Tris Base, 0.1M; NaCl, 0.15 M, pH8)
   Protocol
1. Dilute the antigen to a concentration of 2.5 mg ml⁻¹ in TBS.
2. 2 Dilute carrier protein to a concentration of 2 mg ml⁻¹.
3. Mix carrier and protein in 4:1 mass ratio in a beaker with a magnetic stirring bar.
4. Add Glutaraldehyde solution 1 volume per 2.4 volumes of protein solution. Add the glutaraldehyde solution to the continuously stirred protein over a period of 20 min and continue stirring for 90 min at room temperature.
5. Dialyse the reaction mixture against 2000 volumes of TBS for 16 h at 4 degrees C.
   Protocol for Immunization with a Soluble Protein Available in Quantity
   Materials
5 six week old female Balb/c mice.
Approx. 250 micrograms of the antigen.
Sterile isotonic saline or phosphate buffered saline pH7.
10 microgram vial of GMPD adjuvant (GERBU adjuvant 10, GERBU Biotechnik, GmbH, Gailberg, Germany).
1 ml syringe and a 27 gauge injection needle.
1. Resuspend 250 micrograms of the antigen in 1 ml of the aqueous solution.
2. Transfer the resuspended antigen to a 10 microgram vial of GERBU adjuvant and agitate to dissolve the adjuvant.
3. Take the antigen into a 1 ml syringe. Tap the syringe with the nozzle facing upwards in order to dislodge bubbles from the internal surface of the syringe. Attach the needle to the syringe and depress the piston to check that the solution flows through the needle.
4. A single injection of 200 microliters of the solution is made into the intraperitoneal cavity.
   Schedule for Making Monoclonal Antibodies
1. Prepare antigen and develop screening assay.
2. Immunize animals, a minimum of two for each antigen.
3. One week before fusion, thaw myeloma cells and scale-up. About 10⁸ cells for every 10⁸ mouse cells (one spleen) to be fused.
4. Reimmunize the animals 3-4 days before fusion.
5. Split myeloma cells 1:1 with fresh medium on the day before fusion.
6. Fuse cells, plate out in HT medium (hypoxanthine, thiamine medium).
7. After 24 hours carefully replace the HT medium with HAT medium (hypoxanthine, aminopterin and thymidine medium).
8. Seven days after fusion feed cells with HT medium.
9. About 7 days later, reefed with HT medium.
10. Test supernatants from wells with colonies, as the supernatant turns yellow and the cells are about 50-90% confluent.
11. Clone positive wells and reefed with HT medium.
12. Test clones.
13. Scale-up and cryopreserve positive clones.
   Methods of Antigen Isolation

Various methods of antigen isolation exist and are appropriate for the purposes of generating the antibodies of the present invention. The art of selecting and isolating antigen is well described in the literature.

The foregoing detailed description of the preferred embodiments of the invention has been provided for the purposes of illustration and description, and is not intended to be exhaustive or to limit the invention to the precise embodiments disclosed. The embodiments were chosen and described in order to best explain the principles of the invention and its practical application, thereby enabling others skilled in the art to understand the invention for various embodiments and with various modifications as are suited to the particular use contemplated. It is intended that the scope of the invention cover various modifications and equivalents included within the spirit and scope of the appended claims.

1. A method of changing the appearance of color of an eye, comprising:
administering a coloring agent coupled to an antibody to an eye of a subject; and
binding the antibody to the cornea of the eye to change the appearance of color of the eye.

2. A method according to claim 1, wherein said administering comprises administering the coloring agent and coupled antibody to a human subject.

3. A method according to claim 1, further comprising selecting a monoclonal antibody as the antibody administered to the eye of the subject.

4. A method according to claim 1, wherein said administering comprises a topical administration.

5. A method according to claim 1, wherein the color agent comprises a member selected from dyes, stains, and pigments.

6. A method according to claim 6, further comprising removing a portion of the cornea lying in a pupillary line of sight of the eye.

7. A method according to claim 1, wherein the antibody is engineered to bind to an ocular collagen of the cornea.

8. A method according to claim 1, wherein the antibody is engineered to bind to a protein of the cornea.

9. A method according to claim 1, wherein the antibody is engineered to bind to glycos amino glycans of the cornea.

10. A method according to claim 1, wherein the antibody is engineered to bind proteoglycans of the cornea.
